(19) United States
(12) Patent Application Publication
(10) Pub. No.: US 2021/0147788 A1
(43) Pub. Date: May 20, 2021

(54) ISOLATION DEVICE FOR ADIPOSE-DERIVED STROMAL VASCULAR FRACTION

(71) Applicant: GENZIR TECHNOLOGIES PVT. LTD., Hyderabad (IN)

(72) Inventor: Wasia Rizwani, Hyderabad (IN)

(21) Appl. No.: 16/624,344

(22) PCT Filed: Jun. 21, 2018
(86) PCT No.: PCT/IN2018/050407

§ 371 Date: Dec. 19, 2019

(30) Foreign Application Priority Data
Jun. 21, 2017 (IN) 201741021803

(51) Int. Cl. C12M 1/00; C12M 1/12; C12M 1/06; C12N 5/0775; C12M 1/33; B01D 69/02; B01D 69/06; B01D 71/50; B01D 71/36; B01D 71/56; B01F 7/18

(52) U.S. Cl. CPC C12M 45/04; B01F 2215/0034; C12M 27/02; C12M 45/09; C12N 5/0667; C12M 45/02; B01D 69/02; B01D 69/06; B01D 71/50; B01D 71/36; B01D 71/56; B01F 7/18; B01D 2313/243; B01D 2313/32; B01D 2319/02; B01D 2325/02; C12M 23/06

(57) ABSTRACT

An isolation device for adipose-derived stromal vascular fraction is provided. More particularly the embodiments relates to novel systems, devices, methods and kits for adipose tissue collection and stromal vascular fractions isolation from collected adipose. The devices and systems are used in the field of healthcare/regenerative medicine.

### CROSS-REFERENCE TO RELATED APPLICATIONS

This application claims priority to PCT Application No. PCT/IN2018/050407, having a filing date of Jun. 21, 2018, based on IN 201741021803, having a filing date of Jun. 21, 2017, the entire both contents of which are hereby incorporated by reference.

### FIELD OF TECHNOLOGY

The following relates to isolation devices for adipose-derived stromal vascular fraction. More particularly the following relates to novel systems, devices, methods and kits for adipose tissue collection and stromal vascular fraction (SVF) isolation from collected adipose. The devices can be used in the field of healthcare/regenerative medicine.

### BACKGROUND

Regenerative medicine is an emerging field of medicine. Adipose tissue comprises adipocytes, fat cells and other types of cells collectively known as stromal vascular fraction (SVF). Adipose tissue is an abundant, accessible and rich source of adult stem cells with multi potent properties suitable for tissue engineering and regenerative medical applications. There is an increased interest in Adipose-Derived Stem Cells (ADSCs) from human adipose tissue for tissue engineering applications.

Thus, application of adipose tissue derived stromal vascular fraction (SVF) in the field of healthcare/regenerative medicine is known in the art. In known methods/device adipose tissue is collected by a tissue collector by application of suction and collected tissue is mixed with enzyme for dissociation and then SVF is separated and collected.

U.S. Patent Application Publication No. 2005/0260175A1 (Hedrick et al.) describes automated systems and methods that are used to separate cells from a wide variety of tissues. The system separates regenerative cells, e.g., stem and/or progenitor cells, from adipose tissue. The system uses disaggregation agent i.e. collagenase and also uses filters and centrifugation device.

U.S. Patent Application Publication No. 2015/0004702A1 (Raj et al.) describes an automated system for isolating SVF from the mammalian tissue, wherein the system comprises a plurality of containers for storing buffer solutions, tissue samples and digestive buffers, a tissue processing unit for processing the tissues, a cell concentration unit for receiving the aqueous fraction of tissue from the tissue processing unit, a filter vibrator, a waste collection unit for receiving waste tissues and a control unit to control the operation of the system. This system also uses enzymatic digestion.

U.S. Patent Application Publication No. 2006/0051865A1 (Higgins et al.) describes methods of isolating cells from adipose tissue comprising: (a) subjecting adipose tissue to an electromagnetic, sonic, or other wave energy source; and (b) centrifuging the tissue to form a pellet comprising stem cells. The method is carried out with or without enzymatic digestion of the adipose tissue.

U.S. Patent Application Publication No. 2008/0014181A1 (Ariff et al) disclose an automated cell separation apparatus capable of separating cells from a tissue sample for use in cell therapies and/or tissue engineering, wherein the apparatus includes media and tissue dissociating chemical reservoirs, filters, a cell separator and a perfusion flow loop through a graft chamber which supports a graft substrate or other endovascular device.

U.S. Patent Application Publication No. 2010/0285588A1 (Stubbers et al.) describes a unitary apparatus for isolating cells from adipose tissue including a lipid separation processor with a dispersing head equipped with a plurality of ports and a digestion chamber for dissociation of the constituent cells disposed in adipose tissue. The lipid separating apparatus is useful for the separation of lipids and adipocytes from a mixed cell population. A cell seeding chamber may be attached to the cell isolation apparatus. The components of the apparatus may be packaged in modular kit form.

All these patent literatures mention using adipose tissue from patients for isolating adipose derived stem cells. Most of them mention using collagenase or enzyme mix for disrupting tissue to cells and the apparatus includes tubes with filters, use of centrifuge and temperature control.

Clinical Investigations for the device and clinical trials are not being conducted in India using adipose-derived stem cells. Hence these devices cannot be used in India for clinical or medical use. Their use is limited to research in lab. Devices from outside India are highly expensive and stem cell therapy may not become an accessible treatment to all strata of Indian society.

Adipose stem cell based therapies for many regenerative and other chronic diseases is a very promising therapy but needs clinical trials and validation studies in Indian population. Hence there is a need to introduce an inexpensive yet competitive device for medical needs in India. Thus, there is need of reliable, non-cumbersome and cost effective SVF isolation system/device and method.

The embodiments provide novel reliable, non-cumbersome, cost effective, disposable, single use, microbe-free, user-friendly and economic SVF isolation systems/devices and methods. The novel systems/devices designed in embodiments of the present invention employs enzyme or a mix of enzymes for digestion.

### SUMMARY

Make in India initiative for device making and to introduce adipose stem cell based therapies for many regenerative and other chronic diseases. A very promising therapy but needs clinical trials and validation studies in Indian population, hence the need to introduce an inexpensive yet competitive device for medical needs in India.

An aspect relates to designing and providing novel devices/systems to be used for collection of adipose tissue and isolation of SVF.

A further aspect is to provide a cost effective/economical device/system to be used for collection of adipose tissue and isolation of SVF.

A further aspect is to design and provide a cost effective/economical semi-automatic device/system to be used for collection of adipose tissue and isolation of SVF.

A further aspect is to design and provide a cost effective/economical automatic device/system to be used for collection of adipose tissue and isolation of SVF.

A further aspect is to design and provide an advantageous SVF isolation device/system/method than the existing models.

A further aspect is to design and provide advantageous SVF isolation devices/systems which are disposable, single use device, microbe-free, user-friendly and economical.

Yet in a further aspect the invention the SVF isolation device/system is provided as a kit.

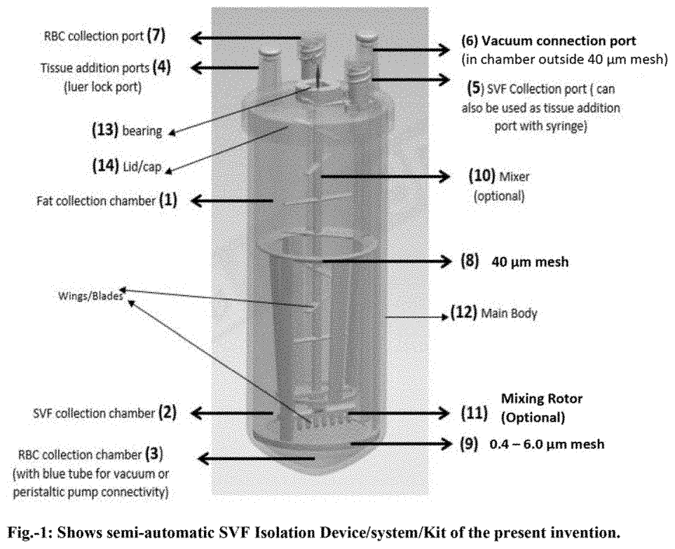

Accordingly, embodiments of the present invention design and provide a novel devices/systems/kits/methods for isolation of SVF from adipose tissue, which is diagrammatically presented in FIG. 1 to FIG. 9. The description of the SVF isolation devices/system and working procedure are described below with reference to Figures.

The embodiments relate to tissue collection and isolation devices for adipose-derived stromal vascular fraction (GenStem-Adipose Device). More particularly embodiments of the invention relates to novel systems, devices, methods and kits for adipose tissue collection and stromal vascular fraction (SVF) isolation from collected adipose. The devices/system/methods can be used in the field of healthcare/regenerative medicine and also in cosmetology for fat transfer/fat implants for enhancement purposes. The embodiments provide novel reliable, non-cumbersome, cost effective and economic SVF isolation systems/devices and methods therefor.

The devices/system of embodiments of the present invention are advantageous SVF isolation devices/systems which are disposable, single use device, microbe-free, user-friendly and yet economical.

The devices of embodiments of the present invention can also be used for fat washing purpose. Fat is used in cosmetology for fat transfer/fat implants for enhancement purposes. Adipose tissue washed off blood is also being used for healing wounds (under trials). So the chamber of the device with mesh size 40-70 μm mesh retain the fat. Excess fluid is drained down with the help of vacuum application using vacuum pump motor or peristaltic pump.

The operation and/or working of the devices/systems of embodiments of the invention may be semi-automatic or automatic to perform desired functions.

In first aspect embodiments of the invention provides a semi-automatic isolation device/system for isolation of adipose-derived stromal vascular fraction (SVF) and method for the device/system.

In second aspect embodiments of the invention provides an automatic isolation device/system for isolation of adipose-derived stromal vascular fraction (SVF) and method for the device/system.

A semi-automatic isolation device for adipose-derived stromal vascular fraction (SVF), the device comprising:

(a) a tubular main body portion (12) having a top lid (14) with ports (4,5,6,7) and creating a hollow cavity inside the tubular container;
(b) collection chambers (1, 2, 3) within the cavity of the tubular container, with tubular connection means with ports (4,5,6,7) for transporting contents in and out from chambers (1,2,3);
(c) filters (8, 9) for filtering;
(d) optionally, mixing rotors (10, 11) for mixing attached from the lid (14) with the help of a bearing (13);
wherein,
the upper chamber (1) is a fat collection chamber comprising the filter (8) to retain and collect fat at chamber (1);
the middle chamber (2) is a SVF collection chamber comprising the filter (9) to retain and collect SVF at chamber (2); and
the lower chamber (3) is Red blood cells collection chamber to collect RBC; and
   wherein,
the chamber (1) is provided with connection to tissue addition port (4) to supply adipose tissue into filter (8) of chamber (1);
the chamber (2) is provided with connection to SVF collection port (5) to collect SVF from Chamber (2) and provided with connection to vacuum connection port (6) to create vacuum in the cavity of the device;
the chamber (3) is provided with connection to RBC Collection Port (7) to collect RBC from chamber (3); and
   wherein,
adipose tissue aspirated into the upper chamber (1) through port (4) is filtered at filter (8) where fat is retained and blood cells, oils and others pass into the middle chamber (2);
filter (9) at middle chamber (2) retains SVF and passes RBC into lower chamber (3), wherein,
isolated SVF is collected from chamber (2) above filter (9) through port (5).

A method for isolation of adipose-derived stromal vascular fraction (SVF), the method involving a device or system comprising:

(a) a tubular main body portion (12) having a top lid (14) with ports (4,5,6,7) and creating a hollow cavity inside the tubular container;
(b) collection chambers (1, 2, 3) within the cavity of the tubular container, with tubular connection means with ports (4,5,6,7) for transporting contents in and out from chambers (1,2,3);
(c) filters (8, 9) for filtering;
(d) optionally, mixing rotors (10, 11) for mixing attached from the lid (14) with the help of a bearing (13);
wherein,
the upper chamber (1) is a fat collection chamber comprising the filter (8) to retain and collect fat at chamber (1);
the middle chamber (2) is a SVF collection chamber comprising the filter (9) to retain and collect SVF at chamber (2); and
the lower chamber (3) is Red blood cells collection chamber to collect RBC; and
   wherein,
the chamber (1) is provided with connection to tissue addition port (4) to supply adipose tissue into filter (8) of chamber (1);
the chamber (2) is provided with connection to SVF collection port (5) to collect SVF from Chamber (2) and provided with connection to vacuum connection port (6) to create vacuum in the cavity of the device;
the chamber (3) is provided with connection to RBC Collection Port (7) to collect RBC from chamber (3); and
   wherein,
adipose tissue aspirated into the upper chamber (1) through port (4) is filtered at filter (8) where fat is retained and blood cells, oils and others pass into the middle chamber (2);
filter (9) at middle chamber (2) retains SVF and passes RBC into lower chamber (3),
   wherein,
isolated SVF is collected from chamber (2) above filter (9) through port (5).

The device is semi-automatic and the device capacity ranges up to 150 ml or 100-120 ml. The tubular container comprising main body (12) and lid (14) is made up of material selected from poly propylene or Poly carbonate. The filter (8) is made up of material selected from nylon or Polytetrafluoroethylene (PTFE) which has mesh size ranging between 30-90 μm to retain and collect fat. The filter (8) has mesh size ranging between 40-70 μm. In one embodiment, filter (8) has mesh size of 40 μm. In another embodiment, filter (8) has mesh size of 70 μm.

The membrane filter (9) is made up of polycarbonate or polyurethane base and has mesh made up of nylon or mixed cellulose esters attached, size ranging between 0.4-12.0 μm to retain and collect SVF. The filter (9) has mesh size ranging between 0.4-6.0 μm.

The adipose tissue, enzyme solution, wash solution, buffer are supplied into the device chambers and fat, SVF, RBC, waste wash solution and waste buffer are removed or collected from the chambers by suitable tubes made up of Ethyl Vinyl Acetate (EVA) or silicone attached to each port (4,5,6,7) with the help of vacuum or peristaltic pump.

The device is operated in conjunction with an assembled centrifuge optionally with mixer and incubator, all in one single set-up and a vacuum pump or all separately.

The device performs fat digestion wherein the fat is digested in chamber (1) by enzyme selected from Collagenase enzyme Type-1, Collagenase enzyme Type-2, Liberase in a concentration between 0.05-1.0% or 0.05-0.5% or 0.05-0.1%.

The device performs washing which uses washing solution 1×PBS (10 mM phosphate buffered saline) plus 1% antibiotic-antimycotic (ABAM) solution and uses 10 mM phosphate buffered saline (PBS).

The device is kept in centrifugation at a rotation speed of 100-150 rpm with condition (i) dry 37° C. incubator with shaking at 100-150 rpm or (ii) 37° C./5% CO₂ incubator.

The device washing is carried out by rinsing fat with Buffer or wash solution 3 times with twice the volume of adipose tissue or fat/lipoaspirate.

In another aspect an automatic isolation device for adipose-derived stromal vascular fraction (SVF) is provided, the device comprising:
area (1) is the upper part of the main body (5) which comprises a sealed bearing (7) and provisions for sampling ports such as Buffer and Enzyme addition port (8) and Lipoaspirate port (9);
area (2) comprises main body (5), optionally mixer/rotor (10) and Strainer Mesh (11);
area (3) comprises Filter (12) for RBC removal and SVF collection, and
area (4) comprises cap (6);
   wherein,
   the main body (5) and a cap (6) both snap fits to form a closed container by a locking system (17) such that, the an outwardly projected ring (15) at the top of the mesh (11) snap fits with the suitable receiving mechanism such as a cavity created by an outwardly projected ring (16) provided in the inner cavity of the body (5), wherein closed container inside comprising strainer mesh (11), RBC removal filter (12), rotor (10);
   wherein,
   ports (8,9,18,19,20) are provided at the top opening of the main body (5) for supply and removal of samples, final product, washing buffers, enzymes, lipoaspirate into and from the device;
   wherein,
   optionally if present, the mixer (10) comprises a shaft (13) with wings or blades (14) for mixing the contents inside the mesh (11);
   wherein,
   fat is filtered and retained at filter (11) and rest blood and oils are passed through the mesh (11) to the filter (12) which filter and retain SVF at the upper side of the filter (12) and allow RBCs to pass through to the cap (6) at area (4),
   wherein,
   SVF is collected from top of the filter (12) in area (3).

In another aspect an method for isolation of adipose-derived stromal vascular fraction (SVF), the method involving an automated device or system comprising:

area (1) is the upper part of the main body (5) which comprises a sealed bearing (7) and provisions for sampling ports such as Buffer and Enzyme addition port (8) and Lipoaspirate port (9);

area (2) comprises main body (5), optionally mixer/rotor (10) and Strainer Mesh (11);

area (3) comprises Filter (12) for RBC removal and SVF collection, and
area (4) comprises cap (6);
   wherein,
   the main body (5) and a cap (6) both snap fits to form a closed container by a locking system (17) such that, the an outwardly projected ring (15) at the top of the mesh (11) snap fits with the suitable receiving mechanism such as a cavity created by an outwardly projected ring (16) provided in the inner cavity of the body (5), wherein closed container inside comprising strainer mesh (11), RBC removal filter (12), rotor (10);
   wherein,
   ports (8,9,18,19,20) are provided at the top opening of the main body (5) for supply and removal of samples, final product, washing buffers, enzymes, lipoaspirate into and from the device;
   wherein,
   optionally if present, the mixer (10) comprises a shaft (13) with wings or blades (14) for mixing the contents inside the mesh (11);
   wherein,
   fat is filtered and retained at filter (11) and rest blood and oils are passed through the mesh (11) to the filter (12) which filter and retain SVF at the upper side of the filter (12) and allow RBCs to pass through to the cap (6) at area (4), wherein,
   SVF is collected from top of the filter (12) in area (3).

In above automated device and method therefor:

The device capacity ranges upto 500 ml or 200-300 ml. The closed container comprising main body (5) and cap (6) is made up of material selected from polyurethane or Polycarbonate.

The filter (11) is made up of material selected from nylon or Polytetrafluoroethylene (PTFE) which has mesh size ranging between 30-90 μm to retain and collect fat. The filter (11) has mesh size ranging between 40-70 μm. In one embodiment, filter (11) has mesh size of 40 μm. In one embodiment, filter (11) has mesh size of 70 μm.

The membrane filter (12) is made up of polycarbonate or polyurethane base topped with filter made of nylon mesh or mixed cellulose esters and has mesh size ranging between 0.4-12.0 μm to retain and collect SVF. The filter (12) has mesh size ranging between 0.4-6.0 μm.

The adipose tissue, enzyme solution, wash solution, buffer are supplied into the device chambers and fat, SVF, RBC, waste wash solution and waste buffer are removed or collected from the chambers by suitable tubes made up of Ethyl Vinyl Acetate (EVA) or silicone attached to each port (8,9,18,19,20) with the help of vacuum or peristaltic pump.

The device is operated in conjunction with processing machines, wherein processing machine includes machines for centrifugation, temperature control, vortexing, vibrating, mixing, and pumping for transporting fluids and other materials. The device performs fat digestion wherein the fat is digested in chamber (1) by enzyme selected from Collagenase enzyme Type-1, Collagenase enzyme Type-2, Liberase in a concentration between 0.05-2.0% or 0.05-1.0% or 0.05-0.5% or 0.05-0.1%.

The device performs washing which uses washing solution 1×PBS (10 mM phosphate buffered saline) plus 1% antibiotic-antimycotic (ABAM) solution and uses 10 mM phosphate buffered saline (PBS).

The device is kept in centrifugation at a rotation speed of 100-150 rpm, optionally with the step of condition (i) dry 37° C. incubator with shaking at 100-150 rpm or (ii) 37° C./5% CO₂ incubator.

The washing is carried out by rinsing fat with Buffer or wash solution 3 times with twice the volume of adipose tissue or fat/lipoaspirate.

Semi-automatic and automatic SVF isolation device as shown and described in FIGS. 1-9.

Method for Semi-automatic and automatic SVF isolation device as shown and described in FIGS. 1-9.

### BRIEF DESCRIPTION

Some of the embodiments will be described in detail, with references to the following Figures, wherein like designations denote like members, wherein:

FIG. 1: Shows a semi-automatic SVF Isolation Device/system/Kit of embodiments of the present invention;

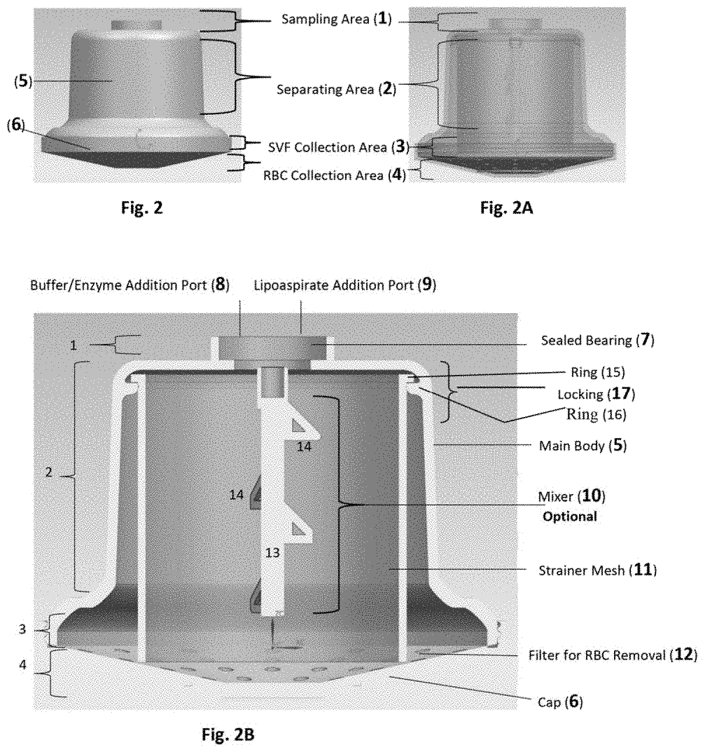

FIG. 2: Shows a front view of the automatic SVF isolation device/system of embodiments of the invention;

FIG. 2A: Shows a transparent view of the device/system as shown in FIG. 2;

FIG. 2B: Shows a cross-sectional view of the device/system of FIG. 2;

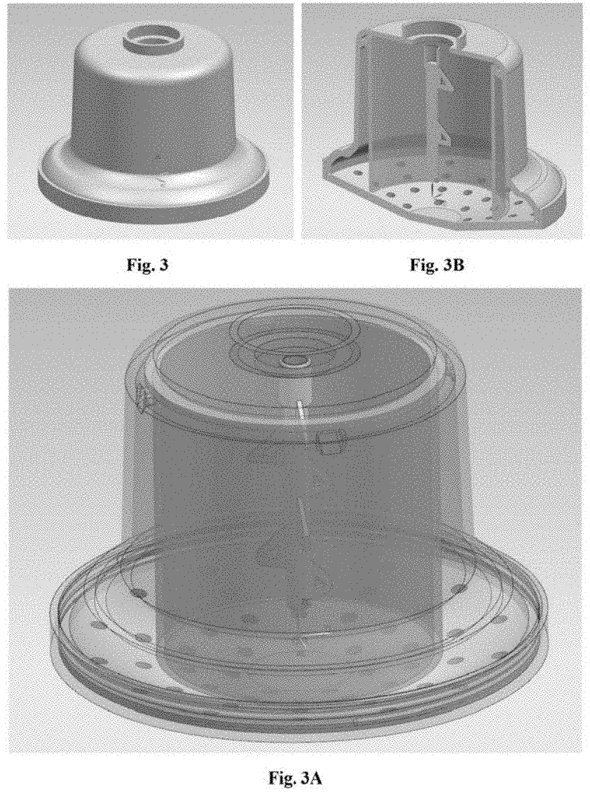

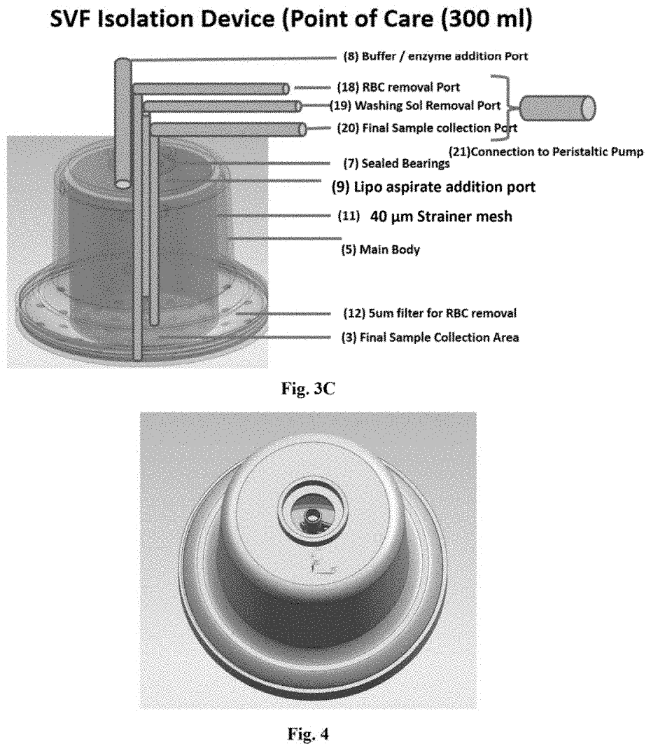

FIG. 3: Shows the isometric view of the automatic SVF isolation device/system;

FIG. 3A: Shows a transparent view of the device/system shown in FIG. 3;

FIG. 3B: Shows a cross-sectional view of the device/system shown in FIG. 3;

FIG. 3C: Shows device/system shown in FIG. 3A with ports;

FIG. 4: Shows a top view of the Device/System;

FIG. 5A: Is an exploded view of the Device/System;

FIG. 5B: Is anexploded view of the Device/System;

FIG. 5C: Is anexploded view of the Device/System;

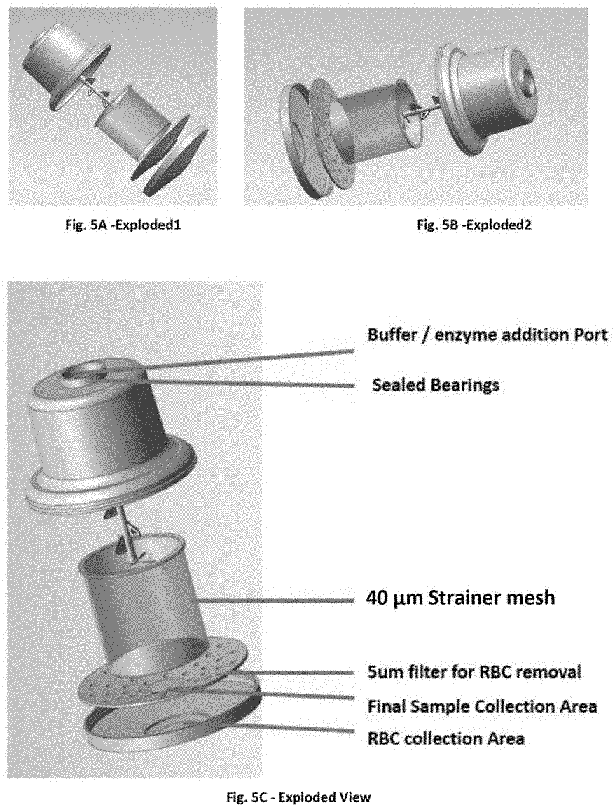

FIG. 6A: Shows a main body of the device/system;

FIG. 6B—Shows a main body of the device/system;

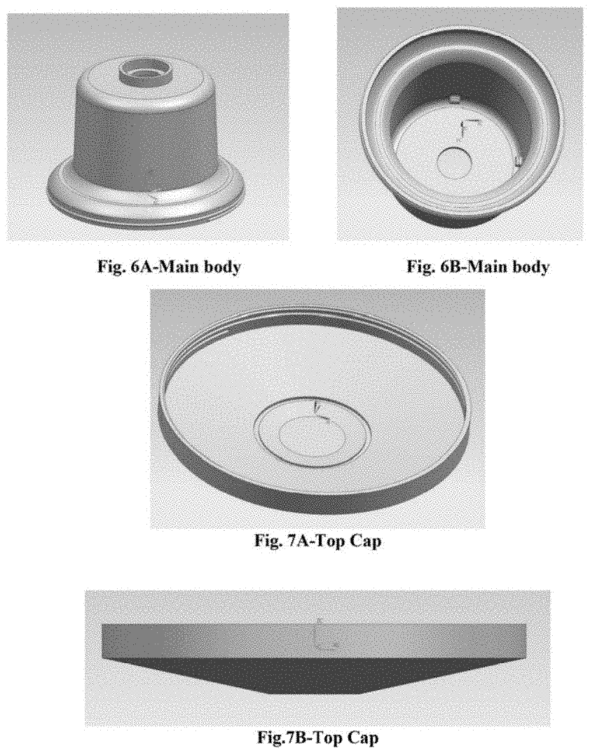

FIG. 7A-Shows a top cap of the device/system;

FIG. 7B-Shows a top cap of the device/system;

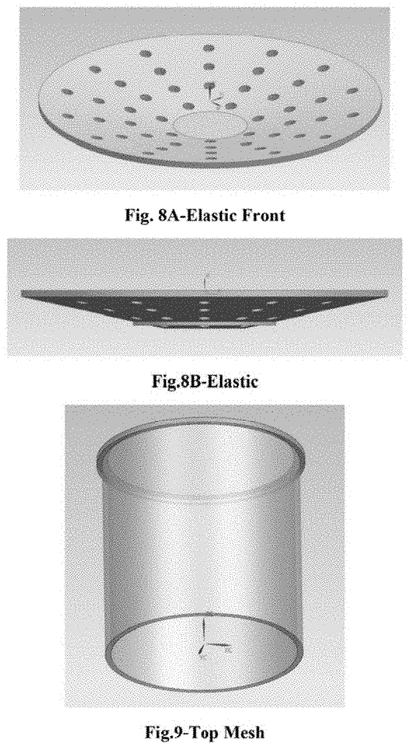

FIG. 8A-Depicts a front view of an elastic filter;

FIG. 8B-Depicts a side view of an elastic filter; and

FIG. 9—Shows a bottom and side view of a top mesh of the device.

### DETAILED DESCRIPTION

Accordingly embodiments of the present invention designs and provides a novel devices/systems/kits/methods for isolation of SVF from adipose tissue, which is diagrammatically presented in FIG. 1 to FIG. 9. The description of the SVF isolation devices/system and working procedure are described below with reference to Figures.

The embodiments relate to tissue collection and isolation devices for adipose-derived stromal vascular fraction (GenStem-Adipose Device). More particularly embodiments of the invention relates to novel systems, devices, methods and kits for adipose tissue collection and stromal vascular fraction (SVF) isolation from collected adipose. The devices/system/methods can be used in the field of healthcare/regenerative medicine and also in cosmetology for fat transfer/fat implants for enhancement purposes. The embodiments provide novel reliable, non-cumbersome, cost effective and economic SVF isolation systems/devices and methods therefor.

The devices/system of embodiments of the present invention are advantageous SVF isolation devices/systems which are disposable, single use device, microbe-free, user-friendly and yet economical.

The devices of embodiments of the present invention can also be used for fat washing purpose. Fat is used in cosmetology for fat transfer/fat implants for enhancement purposes. Adipose tissue washed off blood is also being used for healing wounds (under trials). So the chamber of the device with mesh size 40-70 μm mesh retain the fat. Excess fluid is drained down with the help of vacuum application using vacuum pump motor or peristaltic pump.

The operation and/or working of the devices/systems of embodiments of the invention may be semi-automatic or automatic to perform desired functions.

In first aspect embodiments of the invention provides a semi-automatic isolation device/system for isolation of adipose-derived stromal vascular fraction (SVF) and method for the device/system.

In second aspect embodiments of the invention provides an automatic isolation device/system for isolation of adipose-derived stromal vascular fraction (SVF) and method for the device/system.

### Unit Description-Semi-Automatic

In one aspect embodiments of the invention provides a device and/or system as shown in FIG. 1. The device shown in FIG. 1 can be used for both (a) fat washing and (b) SVF collection and isolation.

In one embodiment, the device of FIG. 1 is an isolation device/system for isolation of ADSC rich, adipose-derived stromal vascular fraction (SVF).

In one embodiment, the device of FIG. 1 is a fat collection device. The tissue collection and SVF isolation unit of embodiments of the present invention is used for adipose tissue collection through lipoaspiration procedure and further processing for SVF isolation. After adipose tissue collection, the collected fat is washed and dissociated using enzymatic or mechanical means for SVF isolation.

Referring FIG. 1, the device/system of embodiments of the invention is closed unit having multiple ports/opening at the top lid/cap (14) of the device for sampling/addition and/or removal of sample, washing solution, buffers, enzyme and/or isolated products and wastes with capacity of handling lipoaspirate up to 150 ml, preferably up to 100 ml. 100-120 ml of pure fat can be collected using this device. In one preferred embodiment a 100 ml capacity device is shown in FIG. 1.

The embodiments use multiple filters. In one embodiment two filters each with different mesh size are used. The first filter comprises pore size of 30-90 μm, preferably 40-70 μm which retains fat (upper chamber) and passes blood and other contents to downward to second filter. The second filter comprises pore size of 0.4-12.0 μm, preferably 0.4-6.0 μm which retains SVF (middle chamber) and passes RBCs and other fluids and/or washing solution to next downward area (lower chamber), thus SVF is isolated at the top of the second filter/mesh at middle chamber, which is then collected in a separate container by using tube and/or pump.

Referring FIG. 1, the device/system of embodiments of the invention comprises a main body part (12) with a lid/cap (14) which is made up of poly propylene or Poly carbonate material. At the top lid (14) of the device, it comprises multiple ports. In one preferred embodiment four ports (4,5,6,7) are provided as shown in FIG. 1. Inside the device, it is hollow cavity which comprises three collection chambers (1,2,3), separated and formed by two filters (8, 9). The device also optionally comprises a mixer (10) in chamber (1) and a mixing rotor (11) at chamber (2). The mixing rotors (10) and (11) are optional and can be provided in a single shaft or separately. In one embodiment the device comprises only rotor (10). In one embodiment the device comprises only rotor (11). In one embodiment the device comprises both the rotors (10) and (11). In one embodiment rotors (10) and (11) are provided in a single rotating shaft which is attached and supported by a bearing (13) fixed with lid (14).

Upper Chamber (1) is fat collection chamber which comprises filter (8) which retains fat (first upper chamber) and passes blood and other contents to downward to second middle chamber known as SVF collection chamber (2) which comprises filter (9) which retains SVF (second middle chamber) and passes RBCs, saline, oils and other fluids to next RBC Collection Chamber (3) (third lower chamber). Thus SVF is separated and retained in chamber (2) and isolated at the top of the filter/mesh (9), which is then collected in a separate container by using tube and/or pump.

Sample can be collected by using syringe or cannula. The device can be directly connected to fat aspiration cannula at one side and vacuum pump at another side. The lipoaspirate can be added into the device via any of the ports (4) and (5). At port (4) the lipoaspirate can be added via a cannula which can be attached with luer fitting at port (4). Alternatively the SVF collection port (5) can also be used for lipoaspirate addition by using a syringe.

Tissue addition port (4) has connection with upper fat collection chamber (1) and through port (4), adipose tissue is provided into chamber (1) where fat is retained by filter (8).

SVF collection port (5) has connection with SVF collection chamber (2) and through port (5) separated SVF is collected. At vacuum connection port (6), vacuum can be created. Port (7) is RBC collection port for collection of RBC from the RBC collection chamber (3). In all the above ports, desired contents are collected by suitable tubing made up of Ethyl Vinyl Acetate (EVA) or silicone attached to each port with the help of vacuum and/or pump such as peristaltic pump.

The unit can be connected with aspiration cannula at one end and vacuum pump at another side through Ethyl Vinyl Acetate (EVA) tubes.

In one embodiment the filter (8) is having pore size ranging in between 30-90 μm, preferably 40-70 μm, most preferably 40 μm in the middle with distance of 10 cm from Bottom. The 40 μm filter (8) is fitted in the middle of the unit is made up of nylon or Polytetrafluoroethylene (PTFE) materials which tolerate the centrifugal force. The 40 μm filter retains the fat in the upper chamber (1) and allows passing of Blood, saline and other oils to chamber (2). The filter is arranged in U shaped frame and inserted in the unit. The U shape of the filter avoids complete clogging of the filter due to fat accumulation.

The device contains the syringe and/or vacuum ports (4,5,6,7) as shown in FIG. 1 for sample and buffer addition and removal.

Vacuum is created in two ways:
(i) by using a vacuum motor pump (no speed control) and
(ii) using a peristaltic pump (with speed control), and

Two vacuum ports are provided, one port (6) in the chamber (2) above 0.4-6.0 μm mesh (9) for washings and another port (7) below the 0.4-6.0 μm mesh (9) for removing RBC and waste.

Thus the RBC collection port (7) is connected with chamber (3), vacuum connection port (6) is connected with chamber (2).

Optionally the device also contains the mixing rotor/mixer (10) for sample mixing during dissociation in chamber 1. The mixing rotor (10) comprises wings/blades to enable mixing and can be provided with the help of a sealed bearing (13) attached at the top lid (14) of the device.

The middle SVF collection chamber (2) is directly connected to the waste collection unit through vacuum pump port (6). The liquids and other smaller junk material pass to the waste collection bin through vacuum pressure applied at port (6). The device also comprises a mixer (10) at chamber (2) for intermittent mixing of the collected lipoaspirate which helps removing the clogging of the filters during the collection and washing.

The middle chamber (2) of the unit is fitted with track etched membrane/filter (9) on the support frame provided therein with pore size of filter mesh made up of nylon or mixed cellulose esters attached, size ranging in between 0.4-12.0 μm, preferably 0.4-6.0 μm. In one preferred embodiment pore size of filter (9) is 0.4 μm. In one preferred embodiment pore size of filter (9) is 5.0 μm. In one preferred embodiment pore size of filter (9) is 6.0 μm. Above this filter is considered as SVF collection chamber/area (2) from where adipose stem cells are collected after fat dissociation and washing procedures. The 0.4-6.0 μm track etched membrane filter (9) is made up of polycarbonate or polyurethane base is fitted in the container 2 cm above from the bottom. The 0.4-6.0 μm membrane filter (9) allows Red Blood Cells to pass through to the bottom into RBC collection Chamber (3) and retains Adipose stem cells which are larger than 0.4-6.0 μm in diameter at above filter (9) in chamber (2).

The retained SVF is collected from SVF collection chamber (2) through special port (5) after mixing in the chamber.

The device is operated in conjunction with an assembled centrifuge optionally with mixer and incubator, all in one single set-up or all separately. A vacuum pump is connected to the set up separately.

In one embodiment, enzyme dissociation is used. The enzyme can be selected from Collagenase enzyme Type-1 and Collagenase enzyme Type-2 or Liberase or other similar enzyme may be used. The concentration of Collagenase enzyme is between 0.05-1.0% or 0.05-0.5% or 0.05-0.1% depending on sample volume and viscosity. The digestion time may be in between 20 min to 1.5 hrs. In one embodiment the digestion time is 1 hr.

The washing solution can be 1×PBS (10 mM phosphate buffered saline) plus 1% antibiotic-antimycotic (ABAM) solution.

The buffer can be 10 mM phosphate buffered saline (PBS).

The centrifugation can be performed at a rotation speed of 100-150 rpm at temperature 37° C.

Two methods can be employed (i) dry 37° C. incubator with shaking at 100-150 rpm or (ii) 37° C./5% CO₂ incubator.

Shaking or rotations are not needed but can be added to the automatic device when larger volumes have to be digested.

The washing is carried out by rinsing fat with Buffer or wash solution 3 times with twice the volume of adipose tissue or fat/lipoaspirate. 0.05%-1.0% collagenase type I or Type II is used.

### Procedure/Method for Semi-Automated Device/System

The SVF isolation device/system as shown in FIG. 1 can process 100-120 ml of lipoaspirate for isolation of SVF rich in stem cells. The device can be directly connected to fat aspiration cannula at one side and vacuum pump at another side. This process avoids independent collection to syringes and transfer to the SVF isolation kit.

When the negative pressure is build up in the device by vacuum pump at port (6), the collected fat aspirate reaches to the device and retains in the upper chamber (1). The upper chamber (1) of the device separates from lower SVF collection chamber (2) with 40-70 um mesh which traps pure fat at chamber (1) and allows water, blood and other liquids to pass onto second SVF collection chamber (2). From SVF collection chamber (2), the vacuum pump at port (6) aspirates the extra liquid remnants to the waste chamber or waste collection bin.

After lipoaspirate collection, the aspirate is washed by addition of washing buffers through ports. The washing solution can be 1×PBS (10 mM phosphate buffered saline) plus 1% antibiotic-antimycotic (ABAM) solution. The buffer can be 10 mM phosphate buffered saline (PBS).

The washing process is appropriately performed by mixing and centrifugation processes. The centrifugation can be performed at a rotation speed of 100-150 rpm at temperature 37° C. Two methods can be employed (i) dry 37° C. incubator with shaking at 100-150 rpm or (ii) 37° C./5% CO₂ incubator.

In one preferred embodiment, (i) dry 37° C. incubator with shaking at 100-150 rpm or (ii) 37° C./5% CO₂ incubator.

The pure fat is then dissociated using enzymes i.e Collagenase, Liberase etc in a concentration range of 0.05%-1.0%. In one preferred embodiment collagenase type I or Type II is used. After addition of enzymes, the fat is mixed through motorized mixer in 37° C. which is maintained in special centrifuge machine. After thorough mixing and incubation, the unit is centrifuged for separation of SVF from undigested fat. The 40-70 um mesh filter (8) allows only cells (Stem cells and Red Blood cells) to pass through and retains tissue junks in the upper chamber (1).

Passed stem cells and RBC reaches to the SVF chamber (2) and lower 0.4-6.0 um track etched membrane (9) allows only RBC to pass through to the bottom RBC chamber (3). Remaining stem cells are retain onto the 0.4-6.0 um membrane (9) due to its larger diameter. The mixer may air this process to perform effective separation of stem cells from Red Blood Cells.

Stem cells retained on the 0.4-6.0 um membrane filter (9) are mixed and washed thoroughly using washing buffers. Finally 5-10 ml of SVF rich in stem cells is collected from SVF collection chamber through port (5) using sterile syringe.

Further the above described SVF isolation device/kit of embodiments of the present invention is illustrated by way of diagrams in FIG. 1.

An Example of Stem Cell Isolation by Using the Device—

(a) Around 25 ml of fat sample was inserted into the prototype filter (8) in chamber (1) using a syringe needle through one of the openings present on the lid (e.g. port (4)).
(b) Collagenase enzyme (0.05% type I in 10 mM PBS/1% ABAM) was added through the same port (4).
(c) The prototype device was kept in the incubator at 37 deg celsius and 5% CO₂ for 1 hour for the tissue to get digested.
(d) The filter (8) with 40 um mesh trapped pure fat at chamber (1) and allows water, blood and other liquids to pass onto second SVF collection chamber (2).
(e) The device was centrifuged at 2000 rpm for 5-10 min and the cells gathered above the 0.4-6.0 μm mesh (9). Cells were washed twice with PBS and collected in 3 ml PBS using a syringe without the needle and transferred to a 15 ml falcon tube. Blue EVA tubes are for vacuum connection, washings were done by connecting the device to either vacuum pump or peristaltic pump (100-300 rotations speed, depending on sample/buffer volume and viscosity).
(f) The cell pellet obtained above mesh (9) following a second centrifugation at 2000 rpm for 10 min was again dissolved in the media (DMEM with 40% PBS and 1% ABAM) and viability test of the cells was done using trypan blue and the number of cells were determined using haemocytometer.

### (DMEM: Dulbecco's Modified Eagle Medium)

(g) 32*10\hat{ }6 cells were obtained from 25 ml fat sample.

### Unit Description-Automatic Device/System

In another aspect embodiments of the invention designs and provides an automatic device and/or system as shown in FIG. 2 to 9. The device designed and shown in FIGS. 2-9 can also be operated in semi-automatic mode. Accordingly in one embodiment, the device as shown in FIGS. 2-9 is fully automatic. In another embodiment, the device as shown in FIGS. 2-9 is semi-automatic.

### Unit Description-Automatic SVF Isolation Device/System

In another aspect embodiments of the invention designs and provides an automatic device and/or system as shown in FIGS. 2 to 9. The device designed and shown in FIGS. 2-9 can also be operated in semi-automatic mode.

Accordingly in one embodiment, the device as shown in FIGS. 2-9 is fully automatic. In another embodiment, the device as shown in FIGS. 2-9 is semi-automatic. In one preferred embodiment, the device/system of FIGS. 2-9 is automatic.

Thus embodiments of the invention provide an automatic device and/or system, method for the device/system and kit for adipose tissue collection and stromal vascular fraction (SVF) isolation from collected adipose.

The device is a small closed container comprising feeding means such as tubing ports for receiving lipoaspirate, buffer, enzyme, wash solution etc. and to remove waste fluids, RBC, waste washing solution and to collect desired SVF.

The device of embodiments of the invention can be of various size and shape. The capacity of the device is not limited, but the capacity and volume of materials to be processed should be of adequate quantity such that, it can be processed and SVF can be isolated from that. Accordingly the size/capacity of the device may vary such as milliliters (ml) to liters (1).

In one embodiment, the device's capacity is between 50-500 ml. In one embodiment, the device shown and described is with a capacity of 300 ml. However this is not limited and size/capacity may vary as per requirements.

In one preferred embodiment, the device's shape is round. However this is not limited and shape may vary and can be modified to other suitable shapes.

The device comprises provision such as mixer with wings or blades inside the container to enable mixing of the components fed into the inner mesh type container. The mixer can be attached inside device container with the help of a bearing fixed in the outer body of the device such as main body of the device, such that the mixer is hanged downward and freely rotate. The mixer can be rotated by supplying power to it or can be rotated as such when it is placed for centrifugation. The shaft of the mixer rotates as such, when the device is placed inside the processing machines and mixes the contents inside.

The device is operated in conjunction with processing machine(s). The processing machine includes machines for centrifugation, temperature control, vortexing, vibrating, mixing, and pumping for transporting fluids and other materials. All these functions can be performed with a single machine or can be with two or more separate machines arranged such that, desired functions of processing of the container type device of embodiments of the invention can be performed.

In one embodiment, the processing machine has centrifugation option with a temperature control through peltier mechanism.

In one embodiment, the processing machine has vortexing option which is used for mixing the components in the device.

In one embodiment, the processing machine has peristaltic pumps which is used for removing washing solutions from the device to the waste bin as per given protocol.

In one embodiment, the processing machine has peristaltic pumps which is used for removing RBCs from the device as per given protocol.

In one embodiment, the processing machine has peristaltic pump which is used for removing desired SVF from the device as per given protocol.

The device comprises a strainer mesh (first filter 11) in the shape of a container with mesh size ranging between 30-90 μm, preferably 40-70 μm, more preferably 60-70 μm mesh size. In one preferred embodiment the first filter's mesh size is 70 μm. In one preferred embodiment the first filter's mesh size is 40 μm. This mesh type container retains the fat in the upper chamber or the mesh container itself and allows passing of blood, saline, and other oils downwards into the next filter.

The next filter (second filter 12) comprises filter with mesh size ranging between 0.4-12.0 μm, or between 0.4-10.0 μm, preferably ranging between 0.4-6.0 μm. In one preferred embodiment the mesh size of the second filter is 0.4 μm. In one preferred embodiment the mesh size of the second filter is 5.0 μm. In one preferred embodiment the mesh size of the second filter is 6.0 μm. This second filter retains SVF, but allows to pass through RBC and collected in a container such as the cap of the device.

The system/device can be automated, that means can operate automatically without requiring activity by an operator in between to proceed to next step. In the automatic device, all the functions starting from sampling to final collection of SVF is performed by machine intelligence and/or based on operating parameter previously set by the operator.

The automatic SVF isolation device/system of embodiments of the present invention is further described by way of drawings/figures.

In one embodiment, the automatic SVF isolation device/system is the device/system as shown in FIG. 2. View of the automatic device/system from different angles, it cross-sectional view, exploded view and its different elements are shown in FIGS. 2a to 9.

### FIG. 2, 2A, 2B:

FIG. 2 shows the front view of the automatic SVF isolation device/system of embodiments of the invention. FIG. 2A is a transparent view of the device shown in FIG. 2 and FIG. 2B is a cross-sectional view of the device/system of FIG. 2. The device as shown in FIG. 2 from outer view, looks like a closed container having inlet port for sampling at top.

The device/system of FIG. 2, 2A, 2B comprises below areas of the device/system
Sampling area (1) at the top of the device,
Separating area (2) at the middle, and
Final sample (SVF) Collection area (3) after separation (2), and
RBC Collection area (4) at the bottom.

The device/system comprises main body (5), which engages with a cap (6) to make the device/system closed and tight fitted keeping or holding other elements inside the closed container type device. Body (5) and cap (6) are made up of material selected from polyurethane or Polycarbonate. The sampling area (1) comprises the provisions of sampling of buffer, enzyme and lipoaspirate into the device/system. The sampling and addition of above buffer, enzyme and lipoaspirate into the device/system can be made by appropriate ports such as inlet/outlet ports with the help of tubing.

Referring FIG. 2B,
area (1) is the upper part of the main body (5) which comprises a sealed bearing (7) and provisions for sampling ports such as Buffer/Enzyme addition port (8) and Lipoaspirate port (9);
area (2) comprises main body (5), optionally mixer/rotor (10) and Strainer Mesh (11);
area (3) comprises Filter (12) for RBC removal and SVF collection, and
area (4) comprises cap (6).

The main body (5) forms the outer main body part which gives the framing support to the structure of the device. Body (5) engages with a Cap (6) forming a closed type container holding inside a strainer mesh (11), mixer (10) and a filter (12).

The mesh (11) is engaged and fixed with the main body (5) by a suitable locking system (17) such that, the an outwardly projected ring (15) at the top of the mesh (11) snap fits with the suitable receiving mechanism such as a cavity created by an outwardly projected ring (16) provided in the inner cavity of the body (5).

The mixer (10), optionally if present comprises a shaft (13) with wings or blades (14) for mixing the contents inside the mesh (11). The sealed bearing (7) present in body (5), engages with the shaft (13) to hang the mixer (10) inside the mesh (11), such that the mixer can be freely rotate to perform mixing of contents inside the mesh (11) container.

The filter (12) is provided below the mesh (11), which receives contents passed through the mesh (11). The function of the filter (12) is to filter and retain SVF at the upper side of the filter (12) and to allow RBCs to pass through to the cap (6) at area (4).

### FIG. 3, 3A, 3B, 3C:

FIG. 3 shows the isometric view, FIG. 3A shows a transparent view shown in FIG. 3, FIG. 3B shows a cross-sectional view, FIG. 3C shows device of FIG. 3A with ports. As shown the mesh (11), optionally the rotor/mixer (10), body (5), cap (6), bearing (7), and other elements of the device as described with reference to FIGS. 2, 2A, and 2B are clearly visible.

In FIG. 3C the connectivity of ports (8, 9, 18, 19, 20) are shown. These ports are inserted/provided through the main opening at the top of the body (5) having bearing (7).

Through lipoaspirate addition port (8), sample fat lipoaspirate is added into the device.

Through buffer/enzyme addition port (8), buffer or enzyme is added into the device.

Through RBC removal port (18), separated RBC is removed from the device.

Through washing solution removal port (19), waste washing solution after washing is removed from the device.

Through SVF collection port (20), separated SVF is removed from the device, which is the desired final product.

Ports (18, 19, 20) are connected with a peristaltic pump (21), which help in removing the content from the device through these ports.

### FIG. 5A, 5B, 5C:

FIG. 5 (A, B, C) shows the exploded view of the device. As shown the main body at top and the cap at last can be combined and fitted in a closed container type device within which all other elements of the device such as rotor/mixer, strainer mesh, RBC removal filter are assembled. The elements as shown in FIG. 2B are same as shown in FIG. 5C.

In FIG. 6A, 6B, the main body (5) is shown. The inside of the body (5) can be visible in FIG. 6B having projections forming ring for engagement of the ring of mesh (11) where snap fit locking is achieved.

FIG. 9 shows the enlarged view of Top Mesh filter (11) as shown in FIG. 2B. The RBC removal filter (12) is an elastic filter having pores as shown in FIGS. 8A and 8B. As shown in FIG. 9, the mesh at top comprises an outward projected projection forming a ring type structure which forms the ring (15) which is engaged with the cavity formed inside the body (5) by a projected ring (16) for snap fitting and locking forming a closed container type device.

The device comprises a strainer mesh 11 (first filter) in area (2) in the shape of a container with mesh size ranging between 30-90 μm, preferably 40-70μm, more preferably 60-70 μm mesh size. In one preferred embodiment the first filter's mesh size is 40 μm or 70 μm. The second filter (12) is RBC removal filter in the area (3) which comprises pore size of 0.4-12.0 μm, preferably 0.4-6.0 μm, most preferably any of 0.4 μm or 4.0 μm or 5.0 μm or 6.0 μm, which retains SVF and passes RBCs and other fluids and/or washing solution to next downward area (4), thus SVF is isolated at the top of the second filter/mesh (12) which is then collected in a separate container by using tube and/or pump.

The main body part (5) with a lid/cap (6) which is made up of poly urethane or Poly carbonate material. At the top of body (5) of the device, it comprises multiple ports. In one preferred embodiment four ports (8, 9, 18, 19, 20) are provided as shown in FIG. 3C. In all the above ports, desired contents are collected by suitable tubing made up of Ethyl Vinyl Acetate (EVA) or silicone attached to each port with the help of vacuum and/or pump such as peristaltic pump.

The unit can be connected with aspiration cannula at one end and vacuum pump at another side through Ethyl Vinyl Acetate (EVA) tubes.

The filter (11) is made up of nylon or Polytetrafluoroethylene (PTFE) materials which tolerate the centrifugal force. The mesh size ranging between 40-70 μm or 70 μm filter retains the fat and allows passing of Blood, saline and other oils to area (3).

Vacuum is created in two ways:
by using a vacuum motor pump (no speed control) and
using a peristaltic pump (with speed control), and
   The elastic membrane filter (12) is made up of polycarbonate has mesh size ranging between 0.4-12.0 μm to retain and collect SVF. In one embodiment the filter (12) has mesh size ranging between 0.4-6.0 μm.

The adipose tissue, enzyme solution, wash solution, buffer are supplied into the device chambers and fat, SVF, RBC, waste wash solution and waste buffer are removed or collected from the chambers by suitable tubes made up of Ethyl Vinyl Acetate (EVA) or silicone attached to each port (8,9,18,19,20) with the help of vacuum or peristaltic pump.

The device performs fat digestion wherein the fat is digested by enzyme selected from Collagenase enzyme Type-1, Collagenase enzyme Type-2, Liberase in a concentration between 0.05-2.0% or 0.05-1.0% or 0.05-0.5% or 0.05-0.1%.

The device performs washing which uses washing solution 1×PBS (10 mM phosphate buffered saline) plus 1% antibiotic-antimycotic (ABAM) solution and uses 10 mM phosphate buffered saline (PBS).

The device is kept in centrifugation at a rotation speed of 100-150 rpm, optionally with the step of condition (i) dry 37° C. incubator with shaking at 100-150 rpm or (ii) 37° C./5% CO₂ incubator. The washing is carried out by rinsing fat with Buffer or wash solution 3 times with twice the volume of adipose tissue or fat/lipoaspirate.

The device is operated in conjunction with processing machines.

### Processing Machine Specification

Processing machine has centrifugation option with a temperature control through peltier mechanism
Vortexing option will be used for mixing the components in the device.
Peristaltic pump will be used for washing solutions from the device to the waste bin as per given protocol

In one embodiment, enzyme dissociation is used. The enzyme can be selected from Collagenase enzyme Type-1 and Collagenase enzyme Type-2 or Liberase or other similar enzyme may be used. The concentration of Collagenase enzyme is between 0.05-2.0% or 0.05-1.0% or 0.05-0.5% or 0.05-0.1% depending on sample volume and viscosity. The digestion time may be in between 20 min to 1.5 hrs. In one embodiment the digestion time is 1 hr.

The washing solution can be 1×PBS (10 mM phosphate buffered saline) plus 1% antibiotic-antimycotic (ABAM) solution.

The buffer can be 10 mM phosphate buffered saline (PBS).

The centrifugation can be performed at a rotation speed of 100-150 rpm at temperature 37° C.

Two methods can be employed (i) dry 37° C. incubator with shaking at 100-150 rpm or (ii) 37° C./5% CO₂ incubator.

Shaking or rotations are not needed but can be added to the automatic device when larger volumes have to be digested.

The washing is carried out by rinsing fat with Buffer or wash solution 3 times with twice the volume of adipose tissue or fat/lipoaspirate. 0.05%-1.0% collagenase type I or Type II is used.

Alternatively, when the automated device of embodiments of the invention can be provided with provision of ultrasonication rods which can be attached in ports for tissue digestion, in case enzyme digestion is not preferred.

### Procedure/Method for Automated Device/System

The SVF isolation device/system as shown in FIGS. 2-9 can process 100-500 ml of lipoaspirate for isolation of SVF rich in stem cells. In Figure a 300 ml device/system is shown.

Lipoaspirate is added into the device through port (9). After lipoaspirate collection, the aspirate is washed by addition of washing buffers through ports (8). The washing solution can be 1×PBS (10 mM phosphate buffered saline) plus 1% antibiotic-antimycotic (ABAM) solution. The buffer can be 10 mM phosphate buffered saline (PBS).

The washing process is appropriately performed by mixing and centrifugation processes. The centrifugation can be performed at a rotation speed of 100-150 rpm at temperature 37° C. Two methods can be employed (i) dry 37° C. incubator with shaking at 100-150 rpm or (ii) 37° C./5% CO₂ incubator.

In one preferred embodiment, (i) dry 37° C. incubator with shaking at 100-150 rpm or (ii) 37° C./5% CO₂ incubator.

The pure fat is then dissociated using enzymes i.e Collagenase, Liberase etc in a concentration range of 0.05%-2.0%. or 0.05%-1.0%. In one preferred embodiment collagenase type I or Type II is used. After addition of enzymes, the fat is mixed through motorized mixer in 37° C. which is maintained in special centrifuge machine. After thorough mixing and incubation, the unit is centrifuged for separation of SVF from undigested fat. The 40-70 um mesh filter (11) allows only cells (Stem cells and Red Blood cells) to pass through and retains tissue junks in the upper area. Passed stem cells and RBC reaches to the SVF collection area and lower 0.4-6.0 um track elastic or RBC removal filter (12) allows only RBC to pass through to the bottom RBC chamber. Remaining stem cells are retain onto the 0.4-6.0 um membrane (12) due to its larger diameter. The mixer may air this process to perform effective separation of stem cells from Red Blood Cells. Stem cells retained on the 0.4-6.0 um elastic filter (12) are mixed and washed thoroughly using washing buffers.

PROTOCOL for Automatic Device
Add Lipoaspirate through the Given port
Add enzymes through port and place the device in the processing Machine for mixing (Machine will have vortexing/vibrating movement)
Connect the tubing to peristaltic pump
Set the Temperature control at 37° C.
Incubate the device for 2 hrs for dissociation
Centrifuge the device for 5 min at 3000 rpm
Remove all enzyme solution from the Mesh/Strainer through port using peristaltic machine
Add required washing solution through buffer port
Mix them using vortex option in the machine and centrifuge for settling the cells
Remove the RBC and upper layer solution leaving the middle layer in the device (contains stromal vascular fraction SVF)
Repeat the steps for 2-3 times
Finally collect the SVF from middle 5.0 um mesh.

### Advantage

The device of embodiments of the present invention has the following advantages over the existing models:

(1) totally closed system though it is semi-automated device in one aspect of embodiments of the invention;
(2) non-cumbersome;
(3) highly economic yet competitive,
(4) disposable, single use device, microbe-free, user-friendly.

### INDUSTRIAL APPLICATIONS

The industrial applications of embodiments of the present invention include use of the product in isolating clean and consistent adipose-derived mix of stromal vascular cells with a predominant stem cell population. The device has applications in vast array of research covering regenerative medicine, chronic diseases, cancer etc and can be used for clinical trials. ADSCs can be used for treatments of ailments of regenerative, cosmetic and chronic in nature. ADSCs can be sub-cultured and stored for future use in autologous or allogenic therapies. Spent media can be used for isolating growth factors that have varied applications. Stem cells can be used for 3D-bioprinting tissues or organs for research purpose initially and later on for medical use upon FDA approval.

Although the invention has been illustrated and described in greater detail with reference to the preferred exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. An automatic or semi-automatic isolation device for adipose-derived stromal vascular fraction, wherein the semi-automatic device comprises:
(a) a tubular container have a tubular main body portion having a top lid with ports and creating a hollow cavity inside the tubular container;
(b) collection chambers within the cavity of the tubular container, with tubular connectors with ports for transporting contents into and from chambers;
(c) filters for filtering;
(d) mixing rotors for mixing attached from the lid with the help of a bearing;
wherein,
the upper chamber is a fat collection chamber comprising the filter to retain and collect fat at chamber;
the middle chamber is a SVF collection chamber comprising the filter to retain and collect SVF at chamber; and
the lower chamber is a Red blood cells collection chamber to collect RBC; and
wherein,
the chamber is provided with connection to tissue addition port to supply adipose tissue into filter of chamber;
the chamber is provided with connection to SVF collection port to collect SVF from Chamber and provided with connection to vacuum connection port to create vacuum in the cavity of the device;
the chamber is provided with connection to RBC Collection Port to collect RBC from chamber; and
wherein,
adipose tissue aspirated into the upper chamber through port is filtered at filter where fat is retained and blood cells, oils and others pas into the middle chamber;
filter at middle chamber retains SVF and passes RBC into lower chamber,
wherein,
isolated SVF is collected from chamber above filter through port.

2. The device as claimed in claim 1, wherein the device capacity ranges between 100 ml to 150 ml.

3. The device as claimed in claim 1, wherein the tubular container comprising main body and lid is made of material selected from poly propylene or Poly carbonate.

4. The device as claimed in claim 1, wherein the filter is made of a material selected from nylon or and Polytetrafluoroethylene (PTFE) which has mesh size ranging between 30-90 μm.

5. The device as claimed in claim 4, wherein the filter has mesh size ranging between 40-70 μm.

6. The device as claimed in claim 1, wherein the membrane filter is made of polycarbonate or polyurethane base and has mesh made up of nylon or mixed cellulose esters attached, size ranging between 0.4-12.0 μm.

7. The device as claimed in claim 6, wherein the filter has mesh size ranging between 0.4-6.0 μm.

8. The device as claimed in claim 1, wherein the adipose tissue, enzyme solution, wash solution, buffer are supplied into the device chambers and fat, SVF, RBC, waste wash solution and waste buffer are removed or collected from the chambers by suitable tubes made up of Ethyl Vinyl Acetate (EVA), or silicone attached to each port with the help of vacuum or peristaltic pump.

9. The device as claimed in claim 1, wherein the device is operated in conjunction with an assembled centrifuge with mixer and incubator, all in one single set-up and a vacuum pump or all separately.

10. The device as claimed in claim 1, wherein the device performs fat digestion wherein the fat is digested in chamber by enzyme selected from Collagenase enzyme Type-1, Collagenase enzyme Type-2, Liberase in a concentration between 0.05-1.0%.

11. The device as claimed in claim 1, wherein the device performs washing which uses washing solution 1×PBS (10 mM phosphate buffered saline) plus 1% antibiotic-antimycotic (ABAM) solution and uses 10 mM phosphate buffered saline (PBS).

12. The device as claimed in claim 1, wherein the device is kept in centrifugation at a rotation speed of 100-150 rpm with condition (i) dry 37° C. incubator with shaking at 100-150 rpm or (ii) 37° C./5% CO₂ incubator.

13. The device as claimed in claim 1, wherein the washing is carried out by rinsing fat with Buffer or wash solution 3 times with twice the volume of adipose tissue or fat/lipoaspirate.

14. A method for isolation of adipose-derived stromal vascular fraction (SVF) involving the semi-automatic device as claimed in claim 1,
wherein the method of the semi-automatic device for isolation of SVF comprises:
aspiring adipose tissue into the upper chamber through port, which is filtered at filter where fat is retained and blood cells, oils and others passes into the middle chamber;
retaining SVF at tiller at middle chamber and passing RBC into lower chamber,
wherein,
isolated SVF is collected from chamber above filter through port.

15. An automatic or semi-automatic isolation device for adipose-derived stromal vascular fraction, wherein the automatic device comprises:
area the upper part of the main body comprises a sealed bearing and provisions for sampling ports such as Buffer and Enzyme addition port and Lipoaspirate port;
area comprises main body mixer/rotor and Strainer Mesh;
area comprises Filter for RBC removal and SVF collection, and
area comprises cap;
wherein,
the main body and a cap both snap fits to form a closed container by a locking system such that, the an outwardly projected ring at the top of the mesh snap fits with the suitable receiving mechanism such as a cavity created by an outwardly projected ring provided in the inner cavity of the body, wherein closed container inside comprising strainer mesh, RBC removal filter, rotor;
wherein,
ports are provided at the top opening of the main body for supply and removal of samples, final product, washing buffers, enzymes, lipoaspirate into and from the device;
wherein,
the mixer comprises a shaft with wings or blades for mixing the contents inside the mesh;
wherein,
fat is filtered and retained at filter and rest blood and oils passed through the mesh to the filter which filter and retain SVF at the upper side of the filter and allow RBCs to pass through to the cap at area,
wherein,
SVF is collected from top of the filter in area.

16. The device as claimed in claim 15, wherein the device capacity ranges between 200 ml to 500 ml.

17. The device as claimed in claim 15, wherein the closed container comprising main body and cap is made of material selected from poly urethane or Poly carbonate.

18. The device as claimed in claim 15, wherein the filter & 11 is made of material selected from nylon or Polytetrafluoroethylene (PTFE) which has mesh size ranging between 30-90 μm.

19. The device as claimed in claim 18, wherein the filter has mesh size ranging between 40-70 μm.

20. The device as claimed in claim 15, wherein the membrane filter is made of polycarbonate or polyurethane base topped with filter made of nylon mesh or mixed cellulose esters and has mesh size ranging between 0.4-12.0 μm.

21. The device as claimed in claim 20, wherein the filter has mesh size ranging between 0.4-0.6 μm.

22. The device as claimed in claim 15, wherein the adipose tissue, enzyme solution, wash solution, buffer are supplied into the device chambers and fat, SVF, RBC, waste wash solution and waste buffer are removed or collected from the chambers by suitable tubes made up of Ethyl Vinyl Acetate (EVA) or silicone attached to each port with the help of vacuum or peristaltic pump.

23. The device as claimed in claim 15, wherein the device is operated in conjunction with processing machines, wherein processing machine includes machines for centrifugation, temperature control, vortexing, vibrating, mixing, and pumping for transporting fluids.

24. The device as claimed in claim 15, wherein the device performs fat digestion wherein the fat is digested in chamber by enzyme selected from Collagenase enzyme Type-1, Collagenase enzyme Type-2, Liberase in a concentration between 0.05-0.2%.

25. The device as claimed in claim 15, wherein the device performs washing which uses washing solution 1×PBS (10 mM phosphate buffered saline) plus 1% antibiotic-antimycotic (ABAM) solution and uses 10 mM phosphate buffered saline (PBS).

26. The device as claimed in claim 15, wherein the device is kept in centrifugation at a rotation speed of 100-150 rpm, optionally with the step of condition (i) dry 37° C. incubator with shaking at 100-150 rpm or (ii) 37° C./5% CO₂ incubator.

27. The device as claimed in claim 15, wherein the washing is carried out by rinsing fat with Buffer or wash solution three times with twice the volume of adipose tissue or fat/lipoaspirate.

28. A method for isolation of adipose-derived stromal vascular fraction (SVF) involving the automatic device as claimed in claim 15,
wherein the method of the automatic device for isolation of SVF comprises:
filtering fat and retaining at filter, and
passing rest, blood and oils through the mesh to the filter which filter and retain SVF at the upper side of the filter and allow RBCs to pass through to the cap at area,
wherein
SVF is collected from top of the filter in area.
